(19) United States
(12) Patent Application Publication
(10) Pub. No.: US 2002/0193384 A1
(43) Pub. Date: Dec. 19, 2002

(54) Use of azoles for the preventing skin cancer

(76) Inventors: Bernward Fladung, Odenthal (DE); Harald Enzmann, Wuppertal (DE)

Correspondence Address: Richard S Bullitt, Bayer Corporation, Box 1910, 36 Columbia Road, Morristown, NJ, 07962-1910, US

(21) Appl. No.: 10/149,169

(22) PCT Filed: Dec. 12, 2000
(86) PCT No.: PCT/EP00/12595

(30) Foreign Application Priority Data
Dec. 24, 1999 (DE) 199630526

(51) Int. Cl. A61K 31/496; A61K 31/4178

(52) U.S. Cl. 514/254.07; 514/396

(57) ABSTRACT

Azoles are suitable for the prevention of radiation-induced skin cancer.

The invention relates to the use of azoles for the prevention of skin cancer caused by radiation.

According to their wavelength, UV rays are divided into UV-A rays (320-400 nm, UV-A-I: 340-400 nm, UV-A-II: 320-340 nm) or UV-B rays (280-320 nm). Very generally: the harmful effect of UV rays on the human skin increases with decreasing wavelength and increasing exposure time.

UV rays can cause acute and chronic skin damage, the type of damage depending on the wavelength of the radiation. For example, UV-B radiation can cause sunburn (erythema) ranging to the severest of, skin bums. Decreases in enzyme activities, disturbances of the DNA structure and changes in the cell membrane are also known as harmful effects of UV-B rays. UV-A rays penetrate into the deeper layers of the skin, where they can accelerate the ageing process of the skin. Shorter-wave UV-A-II radiation additionally intensifies the development of sunburn. Moreover, UV-A radiation can trigger phototoxic or photoallergic skin reactions.

In extreme cases, very frequent and unprotected irradiation of the skin with sunlight can lead to medically abnormal changes in the skin ranging to skin cancer.

In the fight against tumours, use is often made of ionizing radiation, in particular X-ray radiation (“radiotherapy”). In this connection, it is not only the affected organ, but inevitably also the skin, which is subjected to radiation exposure, which has a harmful effect and, in the worst case, can induce skin cancer. A composition for the prevention of such radiation damage would be extremely desirable.

Azoles inhibit the growth of normal and cancer cells in vitro and tumour growth in vivo; cf. L.R. Benzaquen et al., J.A. (1995) Nat. Med. 1,534 to 540.

We have now found that azoles are suitable for the prevention of radiation-induced skin cancer. For the purposes of the invention, “radiation-induced” means primarily “UV-induced” and “induced by radiotherapy”.

The invention permits, for example, the preparation of azole-containing sunscreens which inhibit or completely prevent the UV-induced formation of skin cancer, in particular of squamous epithelial carcinomas, basaliomas and malignant melanomas.

The invention thus provides for the use of azoles for the preparation of topical compositions for the prevention of radiation-induced skin cancer.

Preferred azoles for the prevention of skin cancer correspond, for example, to the formula

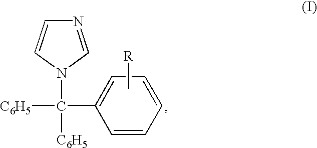

in which

R is a trifluoromethyl, methoxy or o-chlorine substituent, cf. German Auslegeschrift 16 70 976.

Other preferred azoles include e.g.

bifonazole=1-(4-phenylbenzhydryl)-imidazole

butoconazole=(±)-1-[4-(4-chlorophenyl)-2-[(2,6-dichlorophenyl)thio]butyl]-1-H-imidazole

croconazole=1-(1-[2-(3-chlorobenzyloxy)phenyl]vinyl)imidazole

clotrimazole=1-[(2-chlorophenyl)-diphenylmethyl]-1H-imidazole

econazole=1-[2-(4-chlorobenzyloxy)-2-(2,4-dichlorophenyl)-ethyl ]-imidazole

fenticonazole=1-[2-(2,4-dichlorophenyl)-2-[[4-phenylthio)phenyl]methoxy ]-ethyl]-1H-imidazole

fluconazole=2-(2,4-difluorophenyl)-1,3-bis(1H-1,2,4-triazol-1-yl)-2 -propanol

isocanazole=1-[2-(2,4-dichlorophenyl)-2-[(2,6-dichlorophenyl)methoxy ]-ethyl]-1H-imidazole

itraconazole=(±)-2-sec-butyl-4-[4-(4-{[(2R,4S)-2-(2,4-dichlorophenyl)-2 -(1H, 1,2,4-triazol-1-ylmethyl)-1,3-dioxolan-4-yl-methoxy ]-phenyl}-piperazino)-phenyl]-2,4-dihydro-3H-1,2,4-triazol-3 -one

ketoconazole=(±)-1-acetyl-4-{4-[2α-(2,4-dichlorophenyl)-2β-(1 -imidazolylmethyl)-1,3-dioxolan-4β-ylmethoxy]-phenyl}-piperazine

miconazole=(±)-1-[2-(2,4-dichlorobenzyloxy)-2-(2,4-dichlorophenyl)-ethyl]-1H-imidazole

omoconazole=(Z)-1-[2-[2-(4-chlorophenoxy]-2-(2,4-dichlorophenyl)-1 -methylethenyl]-1H-imidazole

oxiconazole=(Z)-1-(2,4-dichlorophenyl)-2-(1H-imidazol-1-yl)ethanone O-[(2,4-dichlorophenyl)methyl]oxime

sertaconazole=(±)-1-[2,4-dichloro-β-[(7-chlorobenzene[b]thien-3-yl)methoxy]-phenethyl]imidazole

sulconazole=1-[2-[[(4-chlorophenyl)methyl]thio]-2-(2,4-dichlorophenyl)ethyl]-1H-imidazole

terconazole=cis-1-[4-[[2-(2,4-dichlorophenyl)-2-(1H-1,2,4-triazol-1 -ylmethyl)-1,3-dioxolan-4-yl]methoxy]phenyl]-4-(1-methylethyl)piperazine

tioconazole=(±)-1-[2-(2-chloro-3-thienylmethoxy)-2-(2,4-dichlorophenyl) -ethyl]-1H-imidazole

The compositions according to the invention can be in the use forms which are customarily used, i.e. e.g. as oil-in-water emulsion or water-in-oil emulsion, as milk, as lotion, cream, aerosol or gel.

The compositions can comprise constituents which are customarily used, such as e.g. emulsifiers, surface-active compounds, lanolin, Vaseline, water, triglycerides of fatty acids, polyethylene glycols, fatty alcohols, ethoxylated fatty alcohols, fatty acid esters (e.g. isopropyl palmitate, isooctyl stearate, diisopropyl adipate etc.), natural or synthetic oils and waxes, pigments (e.g. titanium dioxide, zinc oxide, pearlizing pigments, colour pigments), thickeners (e.g. hydroxyethyl cellulose, bentonite etc.), preservatives, UV absorbers, moisturizers, silicone oils, vitamins, glycerol, ethyl alcohol or perfume oils.

The azoles are generally used in amounts of from 0.3 to 30% by weight, preferably 0.5 to 12% by weight, in particular 1 to 6% by weight, based on the finished preparation (composition).

The compositions according to the invention can be applied and rubbed into the skin prior to radiation exposure. If the irradiation period is relatively long, e.g. in the case of sunbathing, it is advisable to repeat this operation after 2 to 3 hours.

Following close contact with water (bathing, showering), the skin should be completely dried off and the composition according to the invention be rubbed in afresh if radiation exposure is to be continued.

Effectiveness Test

Induction of skin tumours by UV irradiation and reduction thereof on transgenic mice:

Group 1: UV exposure+ optional sun protection

Group 2: UV exposures+ azole+ optional sun protection

End point: as expected, papillomas after 4 to 12 weeks (development of carcinomas requires about 10 months)

The results of the two groups show that azoles protect against UV-induced skin tumours.

1. Use of azoles for the preparation of topical compositions for the prevention of radiation-induced skin cancer.

2. Use according to claim 1, according to which the azoles are chosen from the series bifonazole, butoconazole, clotrimazole, croconazole, econazole, fenticonazole, fluconazole, isoconazole, itraconazole, ketoconazole, miconazole, omoconazole, oxiconazole, sertaconazole, sulconazole, terconazole, tioconazole and mixtures thereof.
